(19) United States
(12) Patent Application Publication
(10) Pub. No.: US 2016/0122296 A1
(43) Pub. Date: May 5, 2016

(54) PROCESS FOR THE OXIDATION OF SULFOXIDES

(71) Applicant: BASF SE, Ludwigshafen (DE)

(72) Inventors: Andrei-Nicolae PARVULESCU, Heidelberg (DE); Ulrich MUELLER, Neustadt (DE); Jan SPIELMANN, Mannheim (DE); Wilfried VOGEL, Dannstadt-Schauernheim (DE); Jun GAO, Neustadt (DE)

(73) Assignee: BASF SE, Ludwigshafen (DE)

(21) Appl. No.: 14/894,432

(22) PCT Filed: May 28, 2014
(86) PCT No.: PCT/EP2014/061097

§ 371 Date: Nov. 27, 2015

(30) Foreign Application Priority Data
May 29, 2013 (EP) 13169755.9

(51) Int. Cl. C07C 315/02; B01J 29/89; C07C 315/06

(52) U.S. Cl. CPC C07C 315/02; C07C 315/06; B01J 29/89

(57) ABSTRACT

The present invention relates to a process for oxidizing a sulfoxide to the respective sulfone, said process comprising reacting the sulfoxide with hydrogen peroxide in the presence of a catalyst, obtaining a mixture (M) comprising the sulfone and the catalyst, wherein the catalyst comprises a porous titanium-containing silicate as a catalytically active material.

The present invention relates to a process for the oxidation of a sulfoxide, which process comprising reacting the sulfoxide with hydrogen peroxide in the presence of a catalyst comprising a porous titanium-containing silicate as a catalytically active material.

Sulfones which are chemical compounds with the general structural formula R₁-S(═O)₂-R₂ where R₁ and R₂ are organic moieties are widely used in chemical industry. For example, diarylsulfones, such as 4,4′-dichlorodiphenylsulfone are important precursors for the production of polyarylene sulfones used, for example, as thermostable polymers.

RU-C-2158257 discloses a process for the preparation of 4,4′-dichlorodiphenyl sulfone comprising reacting, in a first step, thionyl chloride with chlorobenzene in the presence of aluminum chloride to obtain 4,4′-dichlorodiphenyl sulfoxide. In a second step, the sulfoxide is oxidized to 4,4′-dichlorodiphenyl sulfone making use of a mixture comprising hydrogen peroxide and acetic acid.

CN-A-102351757 similarly discloses the synthesis of 4,4′-dichlorodiphenyl sulfone which comprises reacting thionyl chloride with chlorobenzene in the presence of aluminum chloride to 4,4′-dichlorodiphenyl sulfoxide. In a second step, the sulfoxide is oxidized with hydrogen peroxide to 4,4′-dichlorodiphenyl sulfone in the presence of an organoselenic acid as catalyst.

CN-A-102351756 discloses a 4,4′-dichlorodiphenyl sulfone synthesis wherein thionyl chloride is reacted with chlorobenzene in the presence of aluminum trioxide to 4,4′-dichlorodiphenyl sulfoxide. Subsequently, 4,4′-dichlorodiphenyl sulfoxide is oxidized with hydrogen peroxide to 4,4′-dichlorodiphenyl sulfone using heteropolyacids such as phosphotungstic acid and silicotungstic acid supported on activated carbon as catalysts.

In WO-A-2012/143281 a one-pot synthesis for the preparation of a sulfone is disclosed wherein an acid selected from the group consisting of sulfuric acid, arene sulfonic acid and oleum is reacted with fluorinated anhydride and at least one halobenzene. For this reaction, catalysts are disclosed which can be homogeneous or heterogeneous. Among the heterogeneous catalysts, aluminosilicates are described. As aluminosilicates, an H-beta zeolite is described which has a silica : alumina ratio of not more than 40. With regard to the temperature profile, the reaction described in WO-A-2012/143281 is very complex since for individual steps of the reaction, three different temperatures T1, T2 and T3 have to be realized.

CN-A-102838516 discloses a preparation method for sulfoxides and sulfones. As starting materials, thioethers are employed. According to this document, either a sulfoxide or a sulfone is prepared from the thioether. In particular, the document is silent on a process which makes use of a sulfoxide as starting material for the preparation of a sulfone.

In most processes of the prior art, comparatively complex catalyst systems such as supported heteropoly acids or organoselenic acids and/or complex reaction sequences are taught.

Therefore, it was a subject of the present invention to provide an advantageous process for the preparation of a sulfone.

Surprisingly, it was found that such an advantageous process can be realized if a specific heterogeneous catalyst comprising titanium is employed and a sulfoxide is oxidized in the presence of this catayst to obtain the sulfone.

Therefore, the present invention relates to a process for oxidizing a sulfoxide to the respective sulfone, said process comprising
(i) reacting the sulfoxide with hydrogen peroxide in the presence of a catalyst, obtaining a mixture (M) comprising the sulfone and the catalyst,
   wherein the catalyst comprises a porous titanium-containing silicate as a catalytically active material.

### Step (i)

In step (i), a sulfoxide is reacted with hydrogen peroxide in the presence of a catalyst, thereby obtaining a mixture (M) comprising the sulfone and the catalyst, wherein the catalyst comprises a porous titanium-containing silicate as a catalytically active material.

Preferably, the sulfoxide used as educt in step (i) has a structure according to formula (I)

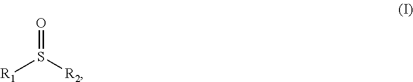

and the respective sulfone obtained as product has a structure according to formula (II)

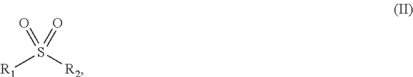

wherein R₁ and R₂ are independently from one another linear or branched, substituted or unsubstituted alkyl residues preferably having from 1 to 20 carbon atoms, linear or branched, substituted or unsubstituted alkenyl residues preferably having from 2 to 20 carbon atoms, or substituted or unsubstituted aryl or heteroaryl residues preferably having from 5 to 20 carbon atoms. Preferably, R₁ and R₂ are independently from one another substituted or unsubstituted aryl residues, more preferably substituted aryl residues. Preferably, the substituents are chosen from the group consisting of halogen such as F, Cl, Br, or I, hydroxyl, linear or branched alkyl residues preferably having from 1 to 10 carbon atoms, linear or branched alkyloxy residues preferably having from 1 to 10 carbon atoms, linear or branched alkenyl residues preferably having from 2 to 10 carbon atoms, aryl residues preferably having from 5 to 10 carbon atoms, heteroaryl residues preferably having from 5 to 10 carbon atoms, and combinations of two or more thereof. Preferably, the heteroatoms of the heteroaryl residues are chosen from the group consisting of N, P, O, and S. More preferably, R₁ and R₂ are independently from one another substituted aryl residues having from 5 to 10 carbon atoms, more preferably from 6 to 10 carbon atoms, wherein the substituents are preferably halogen, such as F, Cl, Br, or I, or hydroxyl, more preferably CI or hydroxyl.

More preferably, the sulfoxide is 4,4′-dichlorodiphenyl sulfoxide according to formula (III). Accordingly, it is preferred that the sulfone obtained in step (i) is 4,4′-dichlorodiphenyl sulfone according to formula (IV):

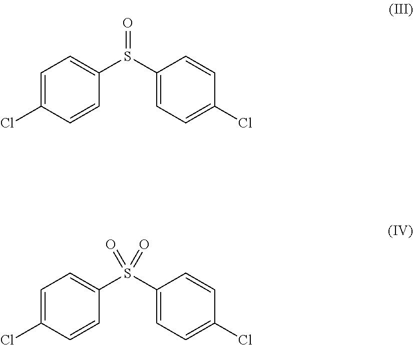 [SMILES: O=S(c1ccc(Cl)cc1)c1ccc(Cl)cc1 ; O=S(=O)(c1ccc(Cl)cc1)c1ccc(Cl)cc1]

Also preferably, the sulfoxide is 4,4′-dihydroxydiphenyl sulfoxide according to formula (IIIa). Accordingly, it is also preferred that the sulfone obtained in step (i) is 4,4′-didihydroxydiphenyl sulfone according to formula (IVa):

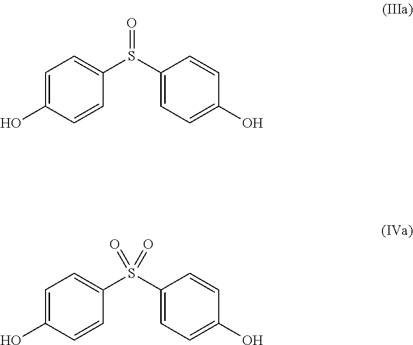 [SMILES: O=S(c1ccc(O)cc1)c1ccc(O)cc1 ; O=S(=O)(c1ccc(O)cc1)c1ccc(O)cc1]

The catalyst used in the process of the present invention comprises a porous titanium-containing silicate as a catalytically active material.

Depending on their pore size, porous silicates can have micropores, i.e. pores having a pore size of less than 2 nanometer, and/or mesopores, e.i. pores having a pore in the range of 2 to 50 nanometer, and/or macropores, i.e. pores having a pore size of more than 50 nanometer. Said pore sizes are to be understood as being determined according to the method as described in DIN 66135 and DIN 66134. Preferably, the porous silicate of the present invention comprises micropores and/or mesopores. More preferably, the porous silicate of the present invention comprises micropores.

Generally, the porous titanium-containing silicate can be amorphous or crystalline. Preferably, the porous titanium-containing silicate is at least partially crystalline. More preferably, at least 50 weight-%, more preferably at least 60 weight-%, more preferably at least 70 weight-%, more preferably at least 80 weight-%, more preferably at least 90 weight-% of the porous titanium-containing silicate are crystalline.

Generally, the porous titanium-containing silicate can comprise the titanium as a constituent of the silicate structure in addition to silicon and oxygen, i.e. as heteroatom in the silicate structure, or as titanium species which is for example adsorbed at or otherwise bound to the silicate structure. Preferably, at least a portion of the titanium is present as a constituent of the silicate structure in addition to silicon and oxygen. More preferably, at least 50%, more preferably at least 60%, more preferably at least 70%, more preferably at least 80%, more preferably at least 90% of the titanium present in the porous titanium-containing silicate is present as a constituent of the silicate structure in addition to silicon and oxygen.

Besides silicon and oxygen and preferably titanium, the silicate framework of the porous silicate may comprise at least one further heteroatom. Conceivable further heteroatoms include, but are no restricted to, Al, Zr, V, Nb, Ta, Cr, Mo, W, Mn, Fe, Co, Ni, Zn, Ga, Ge, In, Sn, Pb. Preferably, the silicate framework of the porous silicate of the present invention is essentially free of aluminum. The term “essentially free of aluminum” as used in this context of the present invention relates to a silicate framework of the porous silicate which comprises 500 ppm or less aluminum, preferably, 300 ppm or less aluminum, more preferably 200 ppm or less aluminum based on the total weight of the silicate framework of the porous silicate. Therefore, more preferred further heteroatoms are selected from the group consisting of Zr, V, Nb, Ta, Cr, Mo, W, Mn, Fe, Co, Ni, Zn, Ga, Ge, In, Sn, Pb, and combinations of two or more thereof.

Preferably, at least 50 weight-%, more preferably at least 60 weight-%, more preferably at least 70 weight-%, more preferably at least 80 weight-%, more preferably at least 90 weight-%, more preferably at least 95 weight-%, more preferably at least 98 weight-%, more preferably at least 99 weight-% of the silicate framework of the porous silicate of the present invention consist of silicon, oxygen, and titanium.

Generally, the porous titanium-containing silicate of the present invention may comprise at least one extra-silicate framework element. Conceivable extra-silicate framework element include, but are no restricted to, Al, Zr, V, Nb, Ta, Cr, Mo, W, Mn, Fe, Co, Ni, Zn, Ga, Ge, In, Sn, Pb. Preferably, the porous titanium-containing silicate of the present invention is essentially free of aluminum. The term “essentially free of aluminum” as used in this context of the present invention relates to a porous titanium-containing silicate which comprises 500 ppm or less aluminum, preferably, 300 ppm or less aluminum, more preferably 200 ppm or less aluminum based on the total weight of the porous titanium-containing silicate. Therefore, more preferred extra-silicate framework element are selected from the group consisting of Zr, V, Nb, Ta, Cr, Mo, W, Mn, Fe, Co, Ni, Zn, Ga, Ge, In, Sn, Pb, and combinations of two or more thereof.

If the porous titanium-containing silicate of the present invention comprises at least one extrasilicate framework element, the at least one element is preferably comprised in an amount in the range of from 0.1 to 10 weight-%, more preferably from 0.2 to 7 weight-%, more preferably from 0.5 to 5 weight-%, based on the total weight of the porous titanium-containing silicate and regarding the sum of all extra-silicate framework elements comprised in the porous titanium-containing silicate.

Preferably, at least a portion of the silicate framework of the titanium-containing porous silicate of the present invention consists of at least one zeolitic framework. More preferably, at least 50 weight-%, more preferably at least 60 weight-%, more preferably at least 70 weight-%, more preferably at least 80 weight-%, more preferably at least 90 weight-%, more preferably at least weight-95%, more preferably at least 98 weight-%, more preferably at least 99 weight-%, more preferably at least 99.9 weight-% of the silicate framework of the titanium-containing porous silicate of the present invention consist of at least one zeolitic framework.

Therefore, the present invention also relates to the process as described above, wherein the porous titanium-containing silicate comprised in the catalyst is a titanium-containing zeolitic material having at least one zeolitic framework structure comprising titanium and silicon.

Preferably, the at least one zeolitic framework structure is selected from the group consisting of ABW, ACO, AEI, AEL, AEN, AET, AFG, AFI, AFN, AFO, AFR, AFS, AFT, AFX, AFY, AHT, ANA, APC, APD, AST, ASV, ATN, ATO, ATS, ATT, ATV, AWO, AWW, BCT, BEA, BEC, BIK, BOG, BPH, BRE, CAN, CAS, CDO, CFI, CGF, CGS, CHA, CHI, CLO, CON, CZP, DAC, DDR, DFO, DFT, DOH, DON, EAB, EDI, EMT, EPI, ERI, ESV, ETR, EUO, FAU, FER, FRA, GIS, GIU, GME, GON, GOO, HEU, IFR, ISV, ITE, ITH, ITQ, ITW, IWR, IWW, JBW, KFI, LAU, LEV, LIO, LOS, LOV, LTA, LTL, LTN, MAR, MAZ, MEI, MEL, MEP, MER, MMFI, MFS, MON, MOR, MSO, MTF, MTN, MTT, MTW, MWW, NAB, NAT, NEES, NON, NPO, OBW, OFF, OSI, OSO, PAR, PAU, PHI, PON, RHO, RON, RRO, RSN, RTE, RTH, RUT, RWR, RWY, SAO, SAS, SAT, SAV, SBE, SBS, SBT, SFE, SFF, SFG, SFH, SFN SFO, SGT, SOD, SSY, STF, STI, STT, TER, THO, TON, TSC, UEI, UFI, UOZ, USI, UTL, VET, VFI, VNI, VSV, WEI, WEN, YNU, YUG, ZON, and mixed structures of two or more thereof. Regarding these three-letter codes and their definitions, reference is made to the “Atlas of Zeolite Framework Types”, 5th edition, Elsevier, London, England (2001).

Preferably, the porous titanium-containing silicate comprised in the catalyst is a titanium-containing zeolitic material having a zeolitic framework structure comprising titanium and silicon.

The zeolitic material which has a zeolitic framework structure comprising titanium and silicon may be produced by substituting titanium into the tetrahedral position of the silicate framework, so that aluminum and/or silicon atoms are at least partly replaced. A zeolitic material having a zeolitic framework structure comprising titanium and silicon, which is preferably aluminum free, can be prepared according to all conceivable methods. In principle, a zeolitic framework structure comprising titanium and silicon can be prepared either by direct synthesis and/or secondary synthesis.

The titanium preferably comprised in the zeolitic framework structure can be incorporated in the framework structure according to all conceivable methods. For example, it is possible to synthesize the zeolitic material based on at least one suitable titanium source, at least one suitably silicon source, and optionally in the presence of at least one suitable template compound. Further, it is conceivable to prepare in a first step a zeolitic material containing a heteroatom other than titanium, such as, for example, aluminum and/or boron, suitably and at least partially remove the heteroatom other than titanium from the zeolitic framework, and introduce titanium in the zeolitic framework at at least a portion of the framework sites previously having been occupied by the heteroatom other than titanium.

In addition to the titanium, the zeolitic framework may include at least one further heteroatom.

Conceivable further heteroatoms include, but are no restricted to, Al, Zr, V, Nb, Ta, Cr, Mo, W, Mn, Fe, Co, Ni, Zn, Ga, Ge, In, Sn, Pb. Preferably, the zeolitic framework of the present invention is essentially free of aluminum. The term “essentially free of aluminum” as used in this context of the present invention relates to a zeolitic framework which comprises 500 ppm or less aluminum, preferably, 300 ppm or less aluminum, more preferably 200 ppm or less aluminum based on the total weight of the zeolitic framework of the zeolitic material. Therefore, more preferred further heteroatoms are selected from the group consisting of Zr, V, Nb, Ta, Cr, Mo, W, Mn, Fe, Co, Ni, Zn, Ga, Ge, In, Sn, Pb, and combinations of two or more thereof.

Preferably, at least 50 weight-%, more preferably at least 60 weight-%, more preferably at least 70 weight-%, more preferably at least 80 weight-%, more preferably at least 90 weight-%, more preferably at least 95 weight-%, more preferably at least 98 weight-%, more preferably at least 99 weight-% of the zeolitic framework of the zeolitic material of the present invention consist of silicon, oxygen, and titanium.

Generally, the porous titanium-containing zeolitic material of the present invention may comprise at least one extra-zeolitic framework element. Conceivable extra-zeolitic framework elements include, but are no restricted to, Al, Zr, V, Nb, Ta, Cr, Mo, W, Mn, Fe, Co, Ni, Zn, Ga, Ge, In, Sn, Pb. Preferably, the porous titanium-containing zeolitic material of the present invention is essentially free of aluminum. The term “essentially free of aluminum” as used in this context of the present invention relates to a porous titanium-containing zeolitic material which comprises 500 ppm or less aluminum, preferably, 300 ppm or less aluminum, more preferably 200 ppm or less aluminum based on the total weight of the titanium-containing zeolitic material. Therefore, more preferred extra-zeolitic framework elements are selected from the group consisting of Zr, V, Nb, Ta, Cr, Mo, W, Mn, Fe, Co, Ni, Zn, Ga, Ge, In, Sn, Pb, and combinations of two or more thereof. According to a conceivable embodiment, the extra-zeolitic framework element includes, preferably is, Zn.

If the porous titanium-containing zeolitic material of the present invention comprises at least one extra-zeolitic framework element, the at least one element is preferably comprised in an amount in the range of from 0.1 to 10 weight-%, more preferably from 0.2 to 7 weight-%, more preferably from 0.5 to 5 weight-%, based on the total weight of the titanium-containing zeolitic material and regarding the sum of all extra-zeolitic framework elements comprised in the titanium-containing zeolitic material.

Preferably, the framework structure of the titanium-containing zeolitic materials comprised in the catalyst used in (i) is selected from the group consisting of MFI, MWW, BEA, MOR, YNU, and a mixed structure of two or more thereof. More preferably, the framework structure of the titanium-containing zeolitic materials comprised in the catalyst used in (i) is selected from the group consisting of MFI and MWW. More preferably, the framework structure of the titanium-containing zeolitic materials comprised in the catalyst used in (i) is not the MFI structure. More preferably, the framework structure of the titanium-containing zeolitic materials comprised in the catalyst is MWW.

Therefore, the present invention also relates to a process for oxidizing a sulfoxide to the respective sulfone, said process comprising
(i) reacting the sulfoxide with hydrogen peroxide in the presence of a catalyst, obtaining a mixture (M) comprising the sulfone and the catalyst,
   wherein the catalyst comprises a titanium-containing zeolitic material as a catalytically active material, wherein the framework structure of the titanium-containing zeolitic materials comprised in the catalyst used in (i) is not the MFI structure.

Further, the present invention relates to a process for oxidizing a sulfoxide to the respective sulfone, said process comprising
(i) reacting the sulfoxide with hydrogen peroxide in the presence of a catalyst, obtaining a mixture (M) comprising the sulfone and the catalyst,
   wherein the catalyst comprises a titanium-containing zeolitic material having framework structure MWW as the catalytically active material.

Therefore, the present also relates to a process for oxidizing the sulfoxide of formula (III)

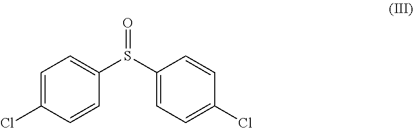 [SMILES: O=S(c1ccc(Cl)cc1)c1ccc(Cl)cc1]

to the respective sulfone of formula (IV)

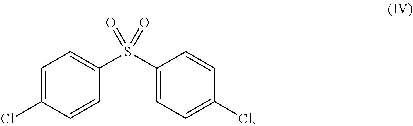 [SMILES: O=S(=O)(c1ccc(Cl)cc1)c1ccc(Cl)cc1]

said process comprising
(i) reacting the sulfoxide with hydrogen peroxide in the presence of a catalyst, obtaining a mixture (M) comprising the sulfone and the catalyst,
   wherein the catalyst comprises a titanium-containing zeolitic material having framework structure MWW as the catalytically active material.

Therefore, the present also relates to a process for oxidizing the sulfoxide of formula (IIIa)

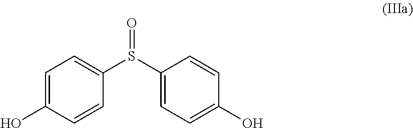 [SMILES: O=S(c1ccc(O)cc1)c1ccc(O)cc1]

to the respective sulfone of formula (IV)

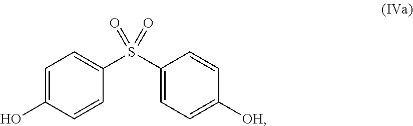 [SMILES: O=S(=O)(c1ccc(O)cc1)c1ccc(O)cc1]

said process comprising
(i) reacting the sulfoxide with hydrogen peroxide in the presence of a catalyst, obtaining a mixture (M) comprising the sulfone and the catalyst,
   wherein the catalyst comprises a titanium-containing zeolitic material having framework structure MWW as the catalytically active material.

Preferably, the MWW framework structure of the titanium-containing zeolitic material comprised in the catalyst has a titanium content in the range of from 0.5 to 3.0 weight-%, preferably from 1 to 2.5 weight-%, more preferably from 1.2 to 2.2 weight-%, calculated as element and based on the total weight of the titanium-containing zeolitic material, and a silicon content in the range of from 30 to 50 weight-%, preferably from 35 to 48 weight-%, more preferably from 38 to 47 weight-%, calculated as element and based on the total weight of the titanium-containing zeolitic material. Therefore, the titanium-containing zeolitic material comprised in the catalyst used in the process of the present invention is a titanium-containing zeolitic material having an MWW framework structure, hereinafter referred to as TiMWW.

Thus, the present invention also relates to a process for oxidizing a sulfoxide to the respective sulfone, said process comprising
(i) reacting the sulfoxide with hydrogen peroxide in the presence of a catalyst, obtaining a mixture (M) comprising the sulfone and the catalyst,
   wherein the catalyst comprises TiMWW as a catalytically active material, preferably as the catalytically active material, wherein the MWW framework structure has a titanium content in the range of from 0.5 to 3.0 weight-%, preferably from 1 to 2.5 weight-%, more preferably from 1.2 to 2.2 weight-%, calculated as element and based on the total weight of the TiMWW.

Further, the present invention relates to a process for oxidizing a sulfoxide to the respective sulfone, said process comprising
(i) reacting the sulfoxide with hydrogen peroxide in the presence of a catalyst, obtaining a mixture (M) comprising the sulfone and the catalyst,
   wherein the catalyst comprises TiMWW as a catalytically active material, preferably as the catalytically active material, wherein the MWW framework structure has a titanium content in the range of from 0.5 to 3.0 weight-%, preferably from 1 to 2.5 weight-%, more preferably from 1.2 to 2.2 weight-%, calculated as element and based on the total weight of the TiMWW, and wherein at least 95 weight-%, preferably at least 98 weight-%, more preferably at least 99 weight-%, more preferably at least 99.5 weight-%, more preferably at least 99.9 weight % of the the MWW framework structure of the titanium-containing zeolitic material comprised in the catalyst consist of Ti, Si, O, and H.

Therefore, the present also relates to a process for oxidizing the sulfoxide of formula (III)

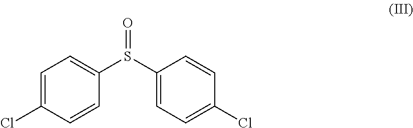 [SMILES: O=S(c1ccc(Cl)cc1)c1ccc(Cl)cc1]

to the respective sulfone of formula (IV)

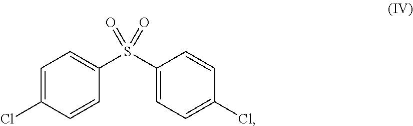 [SMILES: O=S(=O)(c1ccc(Cl)cc1)c1ccc(Cl)cc1]

said process comprising
(i) reacting the sulfoxide with hydrogen peroxide in the presence of a catalyst, obtaining a mixture (M) comprising the sulfone and the catalyst,
   wherein the catalyst comprises TiMWW as a catalytically active material, preferably as the catalytically active material, wherein the MWW framework structure has a titanium content in the range of from 0.5 to 3.0 weight-%, preferably from 1 to 2.5 weight-%, more preferably from 1.2 to 2.2 weight-%, calculated as element and based on the total weight of the TiMWW, and wherein at least 95 weight-%, preferably at least 98 weight-%, more preferably at least 99 weight-%, more preferably at least 99.5 weight-%, more preferably at least 99.9 weight-% of the the MWW framework structure of the titanium-containing zeolitic material comprised in the catalyst consist of Ti, Si, O, and H.

Therefore, the present also relates to a process for oxidizing the sulfoxide of formula (IIIa)

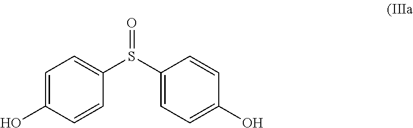 [SMILES: O=S(c1ccc(O)cc1)c1ccc(O)cc1]

to the respective sulfone of formula (IV)

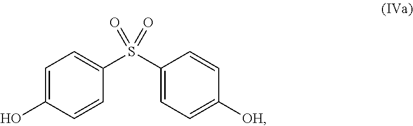 [SMILES: O=S(=O)(c1ccc(O)cc1)c1ccc(O)cc1]

said process comprising
(i) reacting the sulfoxide with hydrogen peroxide in the presence of a catalyst, obtaining a mixture (M) comprising the sulfone and the catalyst,
   wherein the catalyst comprises TiMWW as a catalytically active material, preferably as the catalytically active material, wherein the MWW framework structure has a titanium content in the range of from 0.5 to 3.0 weight-%, preferably from 1 to 2.5 weight-%, more preferably from 1.2 to 2.2 weight-%, calculated as element and based on the total weight of the TiMWW, and wherein at least 95 weight-%, preferably at least 98 weight-%, more preferably at least 99 weight-%, more preferably at least 99.5 weight-%, more preferably at least 99.9 weight-% of the the MWW framework structure of the titanium-containing zeolitic material comprised in the catalyst consist of Ti, Si, O, and H.

The TiMWW may comprise at least one extra-zeolitic framework element. Conceivable extrazeolitic framework elements include, but are no restricted to, Al, Zr, V, Nb, Ta, Cr, Mo, W, Mn, Fe, Co, Ni, Zn, Ga, Ge, In, Sn, Pb. Preferably, the TiMWW is essentially free of aluminum. The term “essentially free of aluminum” as used in this context of the present invention relates to a TiMWW which comprises 500 ppm or less aluminum, preferably, 300 ppm or less aluminum, more preferably 200 ppm or less aluminum based on the total weight of the TiMWW. Therefore, more preferred extra-zeolitic framework elements are selected from the group consisting of Zr, V, Nb, Ta, Cr, Mo, W, Mn, Fe, Co, Ni, Zn, Ga, Ge, In, Sn, Pb, and combinations of two or more thereof. According to a conceivable embodiment, the extra-zeolitic framework element includes, preferably is, Zn.

Preferably, the MWW framework structure of the titanium-containing zeolitic material comprised in the catalyst comprises at most 0.08 weight-% of boron, preferably at most 0.05 weight-% weight-% of boron, calculated as element and based on the total weight of the titanium-containing zeolitic material.

### Preferred Process for the Preparation of TiMWW

Preferably, a zeolitic material of structure type MWW containing titanium (TiMWWW) is prepared in a first step, wherein the obtained TiMWW is optionally subjected in a second step to a suitable treatment to obtain ZnTiMWW.

It is preferred that the TiMWW, optionally further containing zinc, is prepared according to a process comprising
(I) preparing an aluminum-free zeolitic material of structure type MWW containing boron (B-MWW);
(II) deboronating the B-MWW to obtain an aluminum-free zeolitic material of structure type MWW (MWW);
(III) incorporating titanium (Ti) into the MWW to obtain an aluminum-free zeolitic material of structure type MWW containing Ti (TiMWW);
(IV) preferably acid-treating the TiMWW.

### Stage (I)

As far as (I) is concerned, no specific restrictions exist. Preferably, a suitable starting mixture, preferably an aqueous mixture, containing preferably a B containing source and the Si containing source, preferably including at least one suitable micropore-forming agent, is subjected to hydrothermal crystallization under autogenous pressure. For crystallization purposes, it may be conceivable to use at least one suitable seeding material. As suitable Si containing precursors, fumed silica or colloidal silica, preferably colloidal silica such as ammonia-stabilized colloidal silica such as Ludox® AS-40 may be mentioned by way of example. As suitable boron containing precursor, boric acid, B₂O₃, borate salts, preferably boric acid may be mentioned by way of example. As suitable micropore-forming agent, piperidine, hexamethylene imine, or mixtures of piperidine and hexamethylene imine may be mentioned by way of example. Preferably, the crystallization time is in the range of from 3 to 8 days, more preferably from 4 to 6 days. During hydrothermal synthesis, the crystallization mixture may be stirred. The temperatures applied during crystallization are preferably in the range of from 160 to 200° C., more preferably from 160 to 180° C. The B-MMW precursor is obtained in its mother liquor, wherein the mother liquor has preferably a pH above 9.

Preferably, after hydrothermal synthesis, the pH of the mother liquor containing the obtained crystalline zeolitic material B-MMW precursor is adjusted, preferably to a value in the range of from 6 to 9.

The obtained crystalline zeolitic material B-MWW precursor is preferably suitably separated from the mother liquor. All methods of separating the B-MWW precursor from its mother liquor are conceivable. These methods include, for example, filtration, ultrafiltration, diafiltration and centrifugation methods or, for instance, spray drying processes and spray granulation processes. A combination of two or more of these methods can be applied. According to the present invention, the B-MWW precursor is preferably separated from its mother liquid by filtration to obtain a filter cake which is preferably subjected to washing, preferably with water. Subsequently, the filter cake, optionally further processed to obtain a suitable suspension, is subjected to spray drying or to ultrafiltration. Prior to separating the B-MWW precursor from its mother liquor, it is possible to increase the B-MWW precursor content of the mother liquor by concentrating the suspension. If washing is applied, it is preferred to continue the washing process until the washing water has a conductivity of less than 1,000 microSiemens/cm, more preferably of less than 900 microSiemens/cm, more preferably of less than 800 microSiemens/cm, more preferably of less than 700 microSiemens/cm.

After separation of the B-MWW from the suspension, preferably achieved via filtration, and after washing, the washed filter cake containing the B-MWW precursor is preferably subjected to predrying, for example by subjecting the filter cake to a suitable gas stream, preferably a nitrogen stream, for a time preferably in the range of from 4 to 10 h, more preferably from 5 to 8 h.

Subsequently, the pre-dried filter cake is preferably dried at temperatures in the range of from 100 to 300° C., more preferably from 150 to 275° C., more preferably from 200 to 250° C. in a suitable atmosphere such as technical nitrogen, air, or lean air, preferably in air or lean air. Such drying can be accomplished, for example, by spray-drying. Further, it is possible to separate the B-MWW precursor from its mother liquor via a suitable filtration method, followed by washing and spray-drying.

After drying, the B-MWW precursor is preferably subjected to calcination to obtain the B-MWW at temperatures in the range of from 500 to 700° C., more preferably from 550 to 675° C., more preferably from 600 to 675° C. in a suitable atmosphere such as technical nitrogen, air, or lean air, preferably in air or lean air.

### Stage (II)

As far as (II) is concerned, no specific restrictions exist. Preferably, the deboration of the B-MWW to obtain the zeolitic material of structure type MWW (MWW) is achieved via suitable treatment of the B-MWW with a liquid solvent system which may or may not contain at least one inorganic and/or at least one organic acid, or a salt thereof. Conceivable acids are, for example, hydrochloric acid, sulfuric acid, nitric acid, phosphoric acid, formic acid, acetic acid, propionic acid, oxalic acid, and tartaric acid. Preferred acids are inorganic acids, with nitric acid being especially preferred. The liquid solvent system is preferably selected from the group consisting of water, monohydric alcohols, polyhydric alcohols, and mixtures of two or more thereof.

Preferably, the liquid solvent system is selected from the group consisting of water, monohydric alcohols, polyhydric alcohols, and mixtures of two or more thereof, and wherein said liquid solvent system does not contain an inorganic or organic acid or a salt thereof, the acid being selected from the group consisting of hydrochloric acid, sulfuric acid, nitric acid, phosphoric acid, formic acid, acetic acid, propionic acid, and tartaric acid. More preferably, the liquid solvent system does not contain an inorganic or organic acid, or a salt thereof. Even more preferably, the liquid solvent system is selected from the group consisting of water, methanol, ethanol, propanol, ethane-1,2-diol, propane-1,2-diol, propane-1,3-diol, propane-1,2,3-triol, and mixtures of two or more thereof. Most preferably, the liquid solvent system is water.

The treatment according to (II) is preferably carried out at a temperature in the range of from 75 to 125° C., more preferably from 85 to 115° C., for a time preferably in the range of from 8 to 15 h, more preferably from 9 to 12 h.

The obtained deboronated crystalline zeolitic material MWW is preferably suitably separated from the suspension further comprising water and/or acid. All methods of separating the MWW from the suspension are conceivable. These methods include, for example, filtration, ultrafiltration, diafiltration and centrifugation methods or, for instance, spray drying processes and spray granulation processes. A combination of two or more of these methods can be applied. According to the present invention, the MWW is preferably separated from the suspension by filtration to obtain a filter cake which is preferably subjected to washing, preferably with water. Subsequently, the filter cake, optionally further processed to obtain a suitable suspension, is subjected to spray drying or to ultrafiltration. Prior to separating the MWW from the suspension, it is possible to increase the MWW content of the suspension by concentrating the suspension. If washing is applied, it may be preferred to continue the washing process until the washing water has a conductivity of less than 1,000 microSiemens/cm, more preferably of less than 900 microSiemens/cm, more preferably of less than 800 microSiemens/cm, more preferably of less than 700 microSiemens/cm.

After separation of the MWW from the suspension, preferably achieved via filtration, and after washing, the washed filter cake containing the MWW is preferably subjected to pre-drying, for example by subjecting the filter cake to a suitable gas stream, preferably a nitrogen stream, for a time preferably in the range of from 4 to 10 h, more preferably from 5 to 8 h.

Subsequently, the pre-dried filter cake is preferably dried at temperatures in the range of from 100 to 300° C., more preferably from 150 to 275° C., more preferably from 200 to 250° C. in a suitable atmosphere such as technical nitrogen, air, or lean air, preferably in air or lean air. Such drying can be accomplished, for example, by spray-drying. Further, it is possible to separate the MWW from the suspension via a suitable filtration method, followed by washing and spray-drying.

After drying, the MWW can be subjected to calcination at temperatures in the range of from 500 to 700° C., more preferably from 550 to 675° C., more preferably from 600 to 675° C. in a suitable atmosphere such as technical nitrogen, air, or lean air, preferably in air or lean air. Preferably, no calcination is carried out according to (II).

### Stage (III)

As far as (III) is concerned, no specific restrictions exist. Preferably, a suitable starting mixture, preferably an aqueous mixture, containing the MWW and a Ti containing precursor, and preferably containing at least one suitable micropore-forming agent, is subjected to hydrothermal crystallization under autogenous pressure. It may be conceivable to use at least one suitable seeding material. As suitable Ti containing precursor, tetraalkylorthotitanates such as tetrabutylorthotitanate may be mentioned by way of example. As suitable micropore-forming agent, piperidine, hexamethylene imine, or mixtures of piperidine and hexamethylene imine may be mentioned by way of example. Preferably, the crystallization time is in the range of from 4 to 8 days, more preferably from 4 to 6 days. During hydrothermal synthesis, the crystallization mixture may be stirred. The temperatures applied during crystallization are preferably in the range of from 160 to 200° C., more preferably from 160 to 180° C.

After hydrothermal synthesis, the obtained crystalline zeolitic material TiMWW is preferably suitably separated from the mother liquor. All methods of separating the TiMWW from its mother liquor are conceivable. These methods include, for example, filtration, ultrafiltration, diafiltration and centrifugation methods or, for instance, spray drying processes and spray granulation processes. A combination of two or more of these methods can be applied. According to the present invention, the TiMWW is preferably separated from its mother liquid by filtration to obtain a filter cake which is preferably subjected to washing, preferably with water. Subsequently, the filter cake, optionally further processed to obtained a suitable suspension, is subjected to spray drying or to ultrafiltration. Prior to separating the TiMWW from its mother liquor, it is possible to increase the TiMWW content of the mother liquor by concentrating the suspension. If washing is applied, it is preferred to continue the washing process until the washing water has a conductivity of less than 1,000 microSiemens/cm, more preferably of less than 900 microSiemens/cm, more preferably of less than 800 microSiemens/cm, more preferably of less than 700 microSiemens/cm.

After separation of the TiMWW from its mother liquor, preferably achieved via filtration, and after washing, the washed filter cake containing the TiMWW is preferably subjected to pre-drying, for example by subjecting the filter cake to a suitable gas stream, preferably a nitrogen stream, for a time preferably in the range of from 4 to 10 h, more preferably from 5 to 8 h.

Subsequently, the pre-dried filter cake is preferably dried at temperatures in the range of from 100 to 300° C., more preferably from 150 to 275° C., more preferably from 200 to 250° C. in a suitable atmosphere such as technical nitrogen, air, or lean air, preferably in air or lean air. Such drying can be accomplished, for example, by spray-drying to obtain a spray-powder.

In the alternative, the TiMWW is preferably not separated form the mother liquor following the hydrothermal synthesis. Thus, it is preferred that the mother liquor comprising the TiMWW obtained in the hydrothermal synthesis is directly subjected to spray-drying to obtain a spray-powder.

After drying, the TiMWW may be subjected to calcination at temperatures in the range of from 500 to 700° C., more preferably from 550 to 675° C., more preferably from 600 to 675° C. in a suitable atmosphere such as technical nitrogen, air, or lean air, preferably in air or lean air. Preferably, no calcination is carried out according to (III).

### Stage (IV)

Stage (IV) of the process of the present invention preferably serves for reducing the Ti content of the TiMWW as obtained from stage (III), which reduction of the Ti content is preferably achieved by the acid treatment, and preferably also for reducing the carbon content, which reduction of the carbon content is preferably achieved by the calcination as described below. It is noted that according to a conceivable embodiment of the present invention, it may be possible to prepare a TiMWW in stage (III) which already exhibits the desired Ti content. Further, it may be possible in stage (III) to carry out a suitable calcination which results in a carbon content which is low enough so that the respectively obtained TiMWW could be processed further according to stage (V).

Generally, as far as (IV) is concerned, no specific restrictions exist. Preferably, the acid treatment of the TiMWW as obtained according to stage (III) to obtain the finally desired aluminum-free zeolitic material of structure type TiMWW is achieved via suitable treatment of the TiMWW with at least one acid, preferably an inorganic acid, more preferably nitric acid. The treatment according to (IV) is preferably carried out at a temperature in the range of from 75 to 125° C., more preferably from 85 to 115° C., for a time preferably in the range of from 17 to 25 h, more preferably from 18 to 22 h.

After the acid treatment, the obtained crystalline zeolitic material TiMWW is preferably suitably separated from the suspension further comprising an acid. All methods of separating the TiMWW from the suspension are conceivable. These methods include, for example, filtration, ultrafiltration, diafiltration and centrifugation methods or, for instance, spray drying processes and spray granulation processes. A combination of two or more of these methods can be applied. According to the present invention, the TiMWW is preferably separated from the suspension by filtration to obtain a filter cake which is preferably subjected to washing, preferably with water. Subsequently, the filter cake, optionally further processed to obtained a suitable suspension, is subjected to spray drying or to ultrafiltration. Prior to separating the TiMWW from the suspension, it is possible to increase the TiMWW content of the suspension by concentrating the suspension. If washing is applied, it may be preferred to continue the washing process until the washing water has a conductivity of less than 1,000 microSiemens/cm, more preferably of less than 900 microSiemens/cm, more preferably of less than 800 microSiemens/cm, more preferably of less than 700 microSiemens/cm.

After separation of the TiMWW from the suspension, preferably achieved via filtration, and after washing, the washed filter cake containing the TiMWW is preferably subjected to pre-drying, for example by subjecting the filter cake to a suitable gas stream, preferably a nitrogen stream, for a time preferably in the range of from 4 to 10 h, more preferably from 5 to 8 h.

Subsequently, the pre-dried filter cake is preferably dried at temperatures in the range of from 100 to 300° C., more preferably from 150 to 275° C., more preferably from 200 to 250° C. in a suitable atmosphere such as technical nitrogen, air, or lean air, preferably in air or lean air. Such drying can be accomplished, for example, by spray-drying to obtain a spray-powder. Further, it is possible to separate the TiMWW from the suspension via a suitable filtration method, followed by washing and spray-drying.

After drying, the TiMWW is preferably subjected to calcination at temperatures in the range of from 500 to 700° C., more preferably from 550 to 675° C., more preferably from 600 to 675° C. in a suitable atmosphere such as technical nitrogen, air, or lean air, preferably in air or lean air.

The TiMWW obtained in stage (IV) preferably has a titanium content in the range of from 0.5 to 3.0 weight-%, preferably from 1 to 2.5 weight-%, more preferably from 1.2 to 2.2 weight-%, calculated as element and based on the total weight of the titanium-containing zeolitic material, and a silicon content in the range of from 30 to 50 weight-%, preferably from 35 to 48 weight-%, more preferably from 38 to 47 weight-%, calculated as element and based on the total weight of the titanium-containing zeolitic material.

### Conceivable Stage (V)

According to stage (V), the TiMWW preferably obtained according to stage (IV) may be optionally subjected to a suitable Zn treatment. Generally, as far as (V) is concerned, no specific restrictions exist provided that above-defined preferred ZnTiMWW can be obtained having the preferred Zn and Ti content. Most preferably, stage (V) comprises at least one suitable impregnation stage, more preferably at least one wet impregnation stage.

Concerning this impregnation stage, it is preferred to contact the TiMWW preferably as obtained according to (IV) is contacted with at least one suitable Zn-containing precursor in at least one suitable solvent (wet impregnation), most preferably water. As suitable Zn-containing precursor, water-soluble Zn salts are especially preferred, with zinc acetate dihydrate being especially preferred. It is further preferred to prepare a solution of the Zn-containing precursor, preferably an aqueous solution, and to suspend the TiMWW in this solution. Further preferably, impregnation is carried out at elevated temperatures, relative to room temperature, preferably in the range of from 75 to 125° C., more preferably from 85 to 115° C., for a time preferably in the range of from 3.5 to 5 h, more preferably from 3 to 6 h. Stirring the suspension during impregnation is preferred. After the impregnation, the obtained ZnTiMWW is preferably suitably separated from the suspension. All methods of separating the ZnTiMWW from the suspension are conceivable. Especially preferably, separation is carried out via filtration, ultrafiltration, diafiltration or centrifugation methods. A combination of two or more of these methods can be applied. According to the present invention, the ZnTiMWW is preferably separated from the suspension by filtration to obtain a filter cake which is preferably subjected to washing, preferably with water. If washing is applied, it may be preferred to continue the washing process until the washing water has a conductivity of less than 1,000 microSiemens/cm, more preferably of less than 900 microSiemens/cm, more preferably of less than 800 microSiemens/cm, more preferably of less than 700 microSiemens/cm. Subsequently, the preferably washed filter cake is subjected to pre-drying, for example by subjecting the filter cake to a suitable gas stream, preferably a nitrogen stream, for a time preferably in the range of from 5 to 15 h, more preferably from 8 to 12. Preferably, the ZnTiMWW obtained from the impregnation in (V) has a zinc content preferably in the range of from 1.0 to 2.0 weight-%, more preferably from 1.1 to 1.7 weight-%, more preferably from 1.2 to 1.6 weight-%, more preferably from 1.3 to 1.5 weight-%, calculated as elemental zinc, a titanium content in the range of from 0.5 to 3.0 weight-%, preferably from 1 to 2.5 weight-%, more preferably from 1.2 to 2.2 weight-%, calculated as element and based on the total weight of the titanium-containing zeolitic material, and a silicon content in the range of from 30 to 50 weight-%, preferably from 35 to 48 weight-%, more preferably from 38 to 47 weight-%, calculated as element and based on the total weight of the titanium-containing zeolitic material.

### Spray Powder and Molding

The reacting according to step (i) of the process of the present invention can be carried out, for example, in batch mode, in semi-continuous mode, and/or in continuous mode. Depending on the respective mode, it is possible to employ the porous titanium-containing silicate, preferably the titanium-containing zeolitic material, more preferably the TiMWW, as powder. Preferably, if the TiMWW is employed as porous titanium-containing silicate, it is possible to employ the TiMWW as powder, preferably as spray-powder, as obtained according to stage (IV) as described hereinabove.

Preferably, the catalyst used in the process of the present invention is a spray-powder. Optionally, the spray-powder is contained in a molding wherein the molding preferably comprises at least one binder, preferably a silica binder.

Preferably, at least 95 weight-%, preferably at least 98 weight-%, more preferably at least 99 weight-% of the spray powder consist of the porous titanium-containing silicate, preferably the titanium-containing zeolitic material.

Preferably, the spray-powder is present in the form of particles which have a Dv10 value in the range of from 3 to 10 micrometer, preferably from 4 to 6 micrometer, a Dv50 value in the range of from 7 to 50 micrometer, preferably from 8 to 30 micrometer and a Dv90 value in the range of from 12 to 90 micrometer, preferably from 13 to 70 micrometer.

Preferably, the spray powder comprises mesopores having an average pore diameter (4 V/A) in the range of from 10 to 50 nm, preferably from 15 to 45 nm, as determined by Hg porosimetry according to DIN 66133, and comprising macropores having an average pore diameter (4 V/A) in the range of from more than 50 nanometer preferably in the range of from 0.06 to 3 micrometer, as determined by Hg porosimetry according to DIN 66133.

Generally, it is possible to employ the spray powder according to the present invention as such, without any further modifications as a catalyst for the process of the present invention.

It is also possible that based on the spray-powder, a molding is prepared containing the spray-powder. In such a process, the spray-powder, optionally after further modification, is suitably shaped and optionally post-treated. Such further modification of the spray-powder may comprise impregnation of the spray-powder with a solution containing at least one heteroatom, thereby incorporating at least one heteroatom, optionally followed by drying and/or calcining. The molding may be suitably post-treated by incorporating at least one noble metal and/or by subjecting the molding to a water-treatment, wherein the water-treatment comprises treating the molding with liquid water in an autoclave under autogenous pressure at elevated temperatures, followed by optional drying and/or calcination of the molding.

For preparing a molding, the spray-powder used as catalyst in the process of the present invention can be admixed with at least one binder and/or with at least one binder precursor, and optionally with at least one pore-forming agent and/or at least one plasticizing agent.

Examples of suitable binders are metal oxides, such as, for example, SiO₂, Al₂O₃, TiO₂, ZrO₂ or MgO or clays or mixtures of two or more of these oxides or mixed oxides of at least two of Si, Al, Ti, Zr, and Mg. Clay minerals and naturally occurring or synthetically produced alumina, such as, for example, alpha-, beta-, gamma-, delta-, eta-, kappa-, chi- or theta-alumina and their inorganic or organometallic precursor compounds, such as, for example, gibbsite, bayerite, boehmite or pseudoboehmite or trialkoxyaluminates, such as, for example, aluminum triisopropylate, are particularly preferred as Al₂O₃ binders. Further conceivable binders might be amphiphilic compounds having a polar and a non-polar moiety and graphite. Further binders might be, for example, clays, such as, for example, montmorillonites, kaolins, metakaoline, hectorite, bentonites, halloysites, dickites, nacrites or anaxites. Silica binders are especially preferred.

The moldings used in the process of the present invention may contain, based on the weight of the moldings, up to 95 weight-% or up to 90 weight-% or up to 85 weight-% or up to 80 weight% or up to 75 weight-% or up to 70 weight-% or up to 65 weight-% or up to 60 weight-% or up to 55 weight-% or up to 50 weight-% or up to 45 weight-% or up to 40 weight-% or up to 35 weight% or up to 30 weight-% or up to 25 weight-% or up to 20 weight-% or up to 15 weight-% or up to 10 weight-% or up to 5 weight-% of one or more binder materials. Preferably, the moldings of the present invention contain from 10 to 50 weight-%, preferably from 15 to 40 weight-%, more preferably from 20 to 30 weight-% binder, most preferably a silica binder.

Pore forming agents include, but are not limited to, polymers such as polymeric vinyl compounds, such as polyalkylene oxides like polyethylene oxides, polystyrene, polyacrylates, polymethacrylates, polyolefins, polyamides and polyesters, carbohydrates, such as cellulose or cellulose derivatives like methyl cellulose, or sugars or natural fibers. Further suitable pore forming agents may be, for example, pulp or graphite. If desired with regard to the pore characteristics be achieved, a mixture of two or more pore forming agents may be used.

Plasticizing agents include organic, in particular hydrophilic polymers, such as carbohydrate like cellulose, cellulose derivatives, such as methyl cellulose, and starch such as potato starch, wallpaper plaster, polyacrylates, polymethacrylates, polyvinyl alcohol, polyvinylpyrrolidone, polyisobutene or polytetrahydrofuran. The use of water, alcohols or glycols or mixtures thereof, such as mixtures of water and alcohol, or water and glycol, such as for example water and methanol, or water and ethanol, or water and propanol, or water and propylenglycol, as plasticizing agents may be mentioned.

As to the geometry of the moldings used in the process of the present invention, no specific restrictions exist. In particular, the respective geometry may be chosen depending on the specific needs of the specific use of the moldings. When using the molding as catalyst, geometries such as strands, for example having rectangular, triangular hexagonal, quadratic, oval, or circular cross-section, stars, tablets, spheres, hollow cylinders, and the like are possible. One of the preferred geometries of the moldings of the present invention is a strand having circular cross-section. Such geometries are preferred if the moldings of the present invention are employed, for example, as fixed-bed catalysts, most preferably in a continuous-type reaction. The diameter of these strands having circular cross-section which can be prepared, e.g., via extrusion processes, is preferably in a range of from 1 to 4 mm, more preferably from 1 to 3 mm, more preferably from 1 to 2 mm, more preferably from 1.5 to 2 mm, more preferably from 1.5 to 1.7 mm.

For the moldings as catalysts such as fixed-bed catalysts, most preferably in a continuous-type reaction, it is generally necessary that the moldings have superior mechanic resistance in order to allow for a long-term use in the reactor. The molding used in the process of the present invention, preferably in the form of strands having circular cross-section and a diameter of from 1.5 to 1.7 mm, have a crush strength of the least 5 N, preferably a crush strength of up to 20 N, such as from 10 to 20 N, in particular from 11 to 20 N.

### Hydrogen Peroxide

According to the present invention, it is conceivable that the hydrogen peroxide which is used as oxidizing agent is formed in situ during the reaction from hydrogen and oxygen or from other suitable precursors.

Preferably, the hydrogen peroxide is not formed in situ but employed as starting material, preferably in the form of a solution. Preferably, the hydrogen peroxide used in (i) is employed as an aqueous hydrogen peroxide solution. It is further preferred that the aqueous hydrogen peroxide solution has a hydrogen peroxide content in the range of from 10 to 70 weight-%, more preferably from 25 to 60 weight-%, more preferably from 20 to 50 weight-%, based on the total weight of the aqueous solution.

For the preparation of the hydrogen peroxide employed in (i), the anthraquinone process may be used. This process is based on the catalytic hydrogenation of an anthraquinone compound to form the corresponding anthrahydrochinone compound, subsequent reaction of this with oxygen to form hydrogen peroxide and subsequent extraction of the hydrogen peroxide formed. The cycle is completed by rehydrogenation of the anthraquinone compound which has been formed again in the oxidation. A review of the antraquinone process is given in “Ullmanns Encyclopedia of Industrial Chemistry”, 5th edition, volume 13, pages 447 to 456. It is also possible to prepare the hydrogen peroxide by anodic oxidation of sulfuric acid with simultaneous evolution of hydrogen at the cathode to produce peroxodisulfuric acid. Hydrolysis of the peroxodisulfuric acid forms firstly peroxosulfuric acid and then hydrogen peroxide and sulfuric acid, which is thus recovered.

### Reacting in (i)

Preferably, at the beginning of the reaction in (i), the molar ratio of hydrogen peroxide relative to sulfoxide is in the range of from 1:1 to 50:1, more preferably from 2:1 to 30:1, more preferably from 3:1 to 10:1.

Preferably, at the beginning of the reaction in (i), the molar ratio of sulfoxide relative to titanium contained in the titanium-containing silicate, preferably in the framework structure of the titanium-containing zeolitic material, is in the range of from 10:1 to 500:1, more preferably from 30:1 to 300:1, more preferably form 50:1 to 200:1.

Generally, it is conceivable that the reacting in (i) is carried out in the absence of a solvent. Preferably, the reacting in (i) is carried out in the presence of a solvent.

Generally, there are no specific restrictions regarding the chemical nature of the solvent provided that the reacting in (i) can be carried out. Preferably, the solvent is a polar aprotic solvent. More preferably, the solvent is selected from the group consisting of 1-methyl-2-pyrrolidone, tetrahydrofuran, dioxane, chlorinated hydrocarbons, and a mixture of two or more thereof. Chlorinated hydrocarbons are preferably selected from the group consisting of dichloromethane, trichloromethane, trichloroethane, 1,2-dichloroethane, 1,1,2,2-tetrachloroethane, trichloroethylene, 1,2-dichlorobenzene, 1,2,4-trichlorobenzene, and a mixture of two or more thereof. More preferably, the solvent is 1-methyl-2-pyrrolidone.

Consequently, when the reacting in (i) is carried out in the presence of a solvent, the mixture (M) obtained from (i) additionally comprises the solvent.

Preferably, at beginning of the reacting according to (i), the molar ratio of sulfoxide relative to solvent is in the range of from 0.01: 1 to 10:1, preferably from 0.1:1 to 5:1, more preferably from 0.3:1 to 1:1.

Preferably, the reacting according to (i) is carried out in the presence of at least one inert gas. It is conceivable that an inert gas atmosphere comprising at least one inert gas is established above the liquid level at the beginning of the reaction according to (i), whereby no further inert gas is introduced during the reacting in (i). It is also conceivable that the at least one inert gas is introduced continuously into the liquid phase with a suitable flow rate for preferably at least at least partially during the reacting according to (i).

The term “inert gas” as used in this context of the present invention refers to a gas which does not, or not essentially, unfavorably interact with the starting materials, intermediate products or reaction products in the reaction mixture. More preferably, the inert gas is selected from the group consisting of nitrogen, helium, neon, argon, carbon dioxide, and a mixture of two or more thereof. More preferably, the inert gas is nitrogen, more preferably technical nitrogen.

Preferably, the reacting according to (i) is carried out at a temperature in the range of from 0 to 90° C., more preferably from 2 to 85° C., more preferably from 5 to 80° C. A preferred temperature range is from 0 to 20° C., preferably from 2 to 15° C., more preferably from 5 to 10° C. A further preferred temperature range is from 65 to 90° C., preferably from 70 to 85° C., more preferably from 75 to 80° C. Yet a further preferred temperature range is from 30 to 60° C., preferaby from 35 to 55° C., more preferably from 40 to 50° C. It is generally conceivable that during the reaction, two or more suitable different temperatures are applied, provided that these two or more temperatures are within above-mentioned preferred ranges. Heating and/or cooling during the process may be carried out continuously, semi-continuously, or discontinuously. The individual starting materials may be preheated before being mixed together or may be heated following after the mixing.

Preferably, the reaction according to (i) is carried out under a pressure of at most 15 bar, preferably at most 10 bar. It is generally conceivable that during the reacting in (i), two or more suitable different pressures are applied, provided that these two or more pressures are within above-mentioned preferred ranges. Increasing or decreasing the pressure during the process may be carried out continuously, semi-continuously, or discontinuously.

The reacting according to (i) is preferably carried out in batch mode, semicontinuous mode or in continuous mode.

The term “at the beginning of the reaction” as used in the context of the present invention refers to the starting point of the process of the present invention. In batch mode, the “term at the beginning of the reaction” defines the time point at which all educts and the catalyst are present in the educt mixture. In continuous mode which is carried out in a suitable reactor, the “term at the beginning of the reaction” defines the point in the reactor downstream the reactor entrance where the starting materials and the catalyst are for the first time contacted with each other.

The sulfoxide is preferably subjected in (i) in a suitable reactor. Usually, the reactor comprises the heterogeneous catalyst arranged therein and is equipped with means for controlling the reaction pressure, the stirring rate, the inert gas flow, the temperature, and the like. The reactor is further suitable equipped with feeding and removal means. The reactor may be made of materials which are inert under reaction conditions. By way of example, glass or stainless steel may be mentioned.

Preferably, in continuous mode, the catalyst is present in the form of moldings, preferably in the form of strands, arranged in a suitable reactor, for example in the form of a fixed-bed, which enables a thorough contacting with the starting materials which are passed over the catalyst.

Preferably, in batch mode, the titanium-containing zeolitic material as catalytically active material is preferably present as a powder, preferably as a spray-powder, suspended in the liquid starting mixture. The contacting between the zeolitic material and the starting mixture may be enhanced by stirring.

A possible reduction of the activity of the titanium-containing zeolitic material in the course of the oxidation reaction, in particular when performed in continuous mode, may be compensated by adjusting the reaction temperature, pressure, stirring rate, and the like. Indicators for the activity of the titanium-containing zeolitic material are the conversion rate of the educts and the selectivity for the desired product. Conversion rate and selectivity may be calculated according to the formulas indicated below Table 1. Conversion rate and selectivity may be calculated based on the amounts of educts and products present in the reaction mixture at a given time point. The product and educt amounts may be determined by any suitable technique, e.g. chromatography.

When carrying out the reaction according to (i) in a batch mode, it is preferred that the reaction is carried out for a period of time in the range of from 1 to 15 h, preferably from 2 to 10 h, more preferably from 4 to 6 h.

The mixture (M) obtained in (i) following the reaction of sulfoxide with hydrogen peroxide in the presence of a catalyst comprises a sulfone and the catalyst and optionally a solvent. It is conceivable that the mixture (M) obtained in (i) further comprises unreacted starting material and/or one or more by-products.

### Step (ii)

It is preferred that in an additional step (ii), the catalyst is separated from the mixture (M).

The separation of the catalyst may be achieved by any conceivable method. Preferably, in particular in case the reacting in (i) is carried out in batch mode, the catalyst is separated by filtration, centrifugation, draining of mixture (M), pumping out the mixture (M), or a suitable combination of two or more of these methods. More preferably, the catalyst comprising the titanium-containing zeolitic material is separated from the mixture (M) by filtration.

It is conceivable that downstream of the separation of the catalyst according to step (ii), the mixture (M) is directly used, for example without separating the sulfone contained in the mixture (M), as starting material in a suitable reaction. A suitable reaction includes, but is not limited to, a polymerization reaction, wherein the sulfone comprised in mixture (M) is reacted, for example, with one or more suitable compounds such as one or more bifunctional nucleophilic compounds. A suitable polymerization reaction may comprise the preparation of a polyethersulfone such as poly(oxy-1,4-phenylsulfonyl-1,4-phenyl).

Step (iii)

Preferably, the sulfone contained in the mixture (M), preferably the sulfone according to formula (II) contained in the mixture (M), is separated from the mixture (M) in a step (iii).

Preferably, the sulfone, preferably the sulfone according to formula (II), is separated from the mixture (M) by precipitation, crystallization, extraction, solvent evaporation, or a suitable combination of two or more thereof. More preferably the sulfone, preferably the sulfone according to formula (II), is separated from the mixture (M) by precipitation.

It is possible that the sulfone, preferably the sulfone according to formula (II), obtained by separation from mixture (M) is submitted to further purification steps. Further purifications steps may be selected from recrystallization, chromatography, sublimation, or a suitable combination of two or more thereof.

The process of the present invention has considerable advantages over the preparations of sulfones according to the prior art.

In numerous processes of the prior art, oxidizing agents such as peracetic acid and homogeneous catalysts such as Lewis acids are used. Using these compounds require considerable safety precautions so that the sulfone production, particularly at large scale, becomes complex and cost-intensive. Also, by using such acidic compounds considerable amounts of waste water are generated which requires a thorough regeneration before its release in the environment. A further disadvantage of using a homogeneous catalyst is its time- and energy-consuming separation from the product mixture. The process of the present invention has none of these disadvantages, since a substantially inert, heterogeneous catalyst comprising a porous titanium-containing silicate is used, which may be easily separated from the reaction mixture.

Further, the methods of the prior art using heterogeneous catalysts are considerably more complex than the process of the present invention, the former requiring several subsequent reaction stages under highly specific conditions. The educt mixtures of these methods are also complex, further requiring the presence of several different strong acidic compounds in stoichiometric amounts. However, the process of the present invention using a heterogeneous catalyst comprising a porous titanium-containing silicate may be carried out in one single step under constant conditions by reacting merely two educts, a sulfoxide and hydrogen peroxide, to obtain the desired sulfone at favorable yields.

The present invention is further defined by the following embodiments and the combination of embodiments characterized by the respective dependencies:
1. A process for oxidizing a sulfoxide to the respective sulfone, said process comprising
   (i) reacting the sulfoxide with hydrogen peroxide in the presence of a catalyst, obtaining a mixture (M) comprising the sulfone and the catalyst,
      wherein the catalyst comprises a porous titanium-containing silicate as a catalytically active material.
2. The process of embodiment 1, wherein the sulfoxide has a structure according to formula (I)

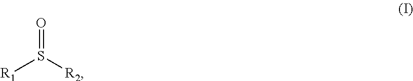

and the respective sulfone has a structure according to formula (II)

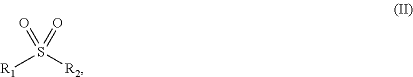

wherein R₁ and R₂ are independently from one another linear or branched, substituted or unsubstituted alkyl residues preferably having from 1 to 20 carbon atoms, linear or branched, substituted or unsubstituted alkenyl residues preferably having from 2 to 20 carbon atoms, or substituted or unsubstituted aryl or heteroaryl residues preferably having from 5 to 20 carbon atoms.
3. The process of embodiment 2, wherein R₁ and R₂ are independently from one another substituted or unsubstituted aryl residues, preferably substituted aryl residues.
4. The process of embodiment 3, wherein the substituents of the aryl residues are selected from the group consisting of halogen, preferably F, Cl, Br, or I, hydroxyl, linear or branched alkyl residues preferably having from 1 to 10 carbon atoms, linear or branched alkyloxy residues preferably having from 1 to 10 carbon atoms, linear or branched alkenyl residues preferably having from 2 to 10 carbon atoms, aryl residues preferably having from 5 to 10 carbon atoms, heteroaryl residues preferably having from 5 to 10 carbon atoms, and combinations of two or more thereof, wherein the heteroatoms of the heteroaryl residues are preferably selected from the group consisting of N, P, O, and S.

5. The process of any of embodiments 2 to 4, wherein R₁ and R₂ are independently from one another substituted aryl residues having from 5 to 10 carbon atoms, preferably from 6 to 10 carbon atoms, wherein the substituents are preferably halogen, more preferably F, Cl, Br, or I, or hydroxyl, more preferably Cl or hydroxyl.
6. The process of any of embodiments 2 to 5, wherein R₁ and R₂ are independently from one another substituted aryl residues having 6, wherein the substituents are Cl or hydroxyl.
7. The process of any of embodiments 1 to 6, wherein the sulfoxide is 4,4′-dichlorodiphenylsulfoxide.
8. The process of any of embodiments 1 to 6, wherein the sulfoxide is 4,4′-dihydroxydiphenylsulfoxide.
9. The process of any of embodiments 1 to 8, wherein the porous titanium-containing silicate comprised in the catalyst is a titanium-containing zeolitic material having a zeolitic framework structure comprising titanium and silicon.
10. The process of embodiment 9, wherein the titanium-containing zeolitic material comprised in the catalyst further comprises one or more elements selected from the group consisting of Al, Zr, V, Nb, Ta, Cr, Mo, W, Mn, Fe, Co, Ni, Zn, Ga, Ge, In, Sn, Pb and a mixture of two or more thereof, the further element preferably being Zn.
11. The process of embodiment 9 or 10, wherein the framework structure of the titanium-containing zeolitic material comprised in the catalyst is an MWW-type framework structure, preferably the MWW framework structure.
12. The process of any of embodiments 9 to 11, wherein the framework structure of the titanium-containing zeolitic material comprised in the catalyst has a titanium content in the range of from 0.5 to 3.0 weight-%, preferably from 1.0 to 2.5 weight-%, more preferably from 1.2 to 2.2 weight-%, calculated as element and based on the total weight of the titanium-containing zeolitic material.
13. The process of any of embodiments 9 to 12, wherein the framework structure of the titanium-containing zeolitic material comprised in the catalyst has a and a silicon content in the range of from 30 to 50 weight-%, preferably from 35 to 48 weight-%, more preferably from 38 to 47 weight-%, calculated as element and based on the total weight of the titanium-containing zeolitic material.
14. The process of any of embodiments 9 to 13, wherein the framework structure of the titanium-containing zeolitic material comprised in the catalyst comprises boron in an amount of from 0 to 0.08 weight-% of boron, preferably from 0 to 0.05 weight-%, calculated as element and based on the total weight of the titanium-containing zeolitic material.
15. The process of any of embodiments 9 to 14, wherein the framework structure of the titanium-containing zeolitic material comprised in the catalyst is the MWW framework structure, wherein the framework structure of the titanium-containing zeolitic material comprised in the catalyst has a titanium content in the range of from 0.5 to 3.0 weight-%, calculated as element and based on the total weight of the titanium-containing zeolitic material, and wherein the framework structure of the titanium-containing zeolitic material comprised in the catalyst has a silicon content in the range of from 30 to 50 weight-%, calculated as element and based on the total weight of the titanium-containing zeolitic material.
16. The process of any of embodiments 9 to 15, wherein the framework structure of the titanium-containing zeolitic material comprised in the catalyst is the MWW framework structure, wherein the framework structure of the titanium-containing zeolitic material comprised in the catalyst has a titanium content in the range of from 1.0 to 2.5 weight-%, calculated as element and based on the total weight of the titanium-containing zeolitic material, and wherein the framework structure of the titanium-containing zeolitic material comprised in the catalyst has a silicon content in the range of from 35 to 48 weight-%, calculated as element and based on the total weight of the titanium-containing zeolitic material.
17. The process of any of embodiments 9 to 16, wherein the framework structure of the titanium-containing zeolitic material comprised in the catalyst is the MWW framework structure, wherein the framework structure of the titanium-containing zeolitic material comprised in the catalyst has a titanium content in the range of from 1.2 to 2.2 weight-%, calculated as element and based on the total weight of the titanium-containing zeolitic material, and wherein the framework structure of the titanium-containing zeolitic material comprised in the catalyst has a silicon content in the range of from 38 to 47 weight-%, calculated as element and based on the total weight of the titanium-containing zeolitic material.
18. The process of any of embodiments 15 to 17, wherein at least 95 weight-%, preferably at least 98 weight-%, more preferably at least 99 weight-%, more preferably at least 99.5 weight-%, more preferably at least 99.9 weight-% of the the MWW framework structure of the titanium-containing zeolitic material comprised in the catalyst consist of Ti, Si, O, and H.
19. The process of any of embodiments 1 to 18, wherein the hydrogen peroxide used in (i) is employed as an aqueous hydrogen peroxide solution, preferably having a hydrogen peroxide content in the range of from 10 to 70 weight-%, more preferably from 15 to 60 weight-%, more preferably from 20 to 50 weight-%, based on the total weight of the aqueous solution.
20. The process of any of embodiments 1 to 19, wherein at the beginning of the reaction according to (i), the molar ratio of hydrogen peroxide relative to sulfoxide is in the range of from 1:1 to 50:1, preferably from 2:1 to 30:1, more preferably from 3:1 to 10:1.
21. The process of any of embodiments 1 to 20, wherein at the beginning of the reaction according to (i), the molar ratio of sulfoxide relative to titanium contained in the titanium-containing silicate, preferably in the framework structure of the titanium-containing zeolitic material, is in the range of from 10:1 to 500:1, preferably from 30:1 to 300:1, more preferably form 50:1 to 200:1.
22. The process of any of embodiments 1 to 21, wherein the reaction according to (i) is carried out in the presence of a solvent and wherein the mixture (M) additionally comprises the solvent.
23. The process of embodiment 22, wherein the solvent is selected from the group consisting of 1-methyl-2-pyrrolidone, tetrahydrofuran, dioxane, chlorinated hydrocarbons, and a combination of two or more thereof.
24. The process of embodiment 22 or 23, wherein at the beginning of the reaction according to (i), the molar ratio of sulfoxide relative to solvent is in the range of from 0.01:1 to 10:1, preferably from 0.1:1 to 5:1, more preferably from 0.3:1 to 1:1.
25. The process of any of embodiments 1 to 24, wherein the reaction according to (i) is carried in the presence of at least one inert gas.
26. The process of embodiment 25, wherein the inert gas is selected from the group consisting of nitrogen, helium, neon, argon, carbon dioxide, and a mixture of two or more thereof, wherein the inert gas more preferably comprises nitrogen, more preferably comprises, more preferably consists of, technical nitrogen.
27. The process of any of embodiments 1 to 26, wherein the reaction according to (i) is carried out at a temperature in the range of from 0 to 90° C., preferably from 2 to 85° C., more preferably from 5 to 80° C.
28. The process of any of embodiments 1 to 27, wherein the reaction according to (i) is carried out under a pressure of at most 15 bar, preferably at most 10 bar.
29. The process of any of embodiments 1 to 28, wherein the reaction according to (i) is carried out under a pressure in the range of from 1 to 15 bar, preferably from 1 to 10 bar.
30. The process of any of embodiments 1 to 29, wherein the reaction according to (i) is carried out in batch mode.
31. The process of embodiment 30, wherein the reaction according to (i) is carried out for a period of time in the range of from 1 to 15 h, preferably from 2 to 10 h, more preferably from 4 to 6 h.
32. The process of any of embodiments 1 to 29, wherein the reaction according to (i) is carried out in continuous mode.
33. The process of any of embodiments 1 to 32, further comprising (ii) separating the catalyst from the mixture (M), preferably by filtration.
34. The process of any of embodiments 1 to 33, further comprising (iii) separating the sulfone according to formula (II) from the mixture (M), preferably by precipitation.
35. The process of any of embodiments 1 to 34, wherein the catalyst is a spray-powder.
36. The process of embodiment 35, wherein at least 95 weight-%, preferably at least 98 weight-%, more preferably at least 99 weight-% of the spray powder consist of the titanium-containing silicate, preferably the titanium-containing zeolitic material.
37. The process of embodiment 35 or 36, wherein the spray-powder is contained in a molding.
38. The process of embodiment 37, wherein the molding comprises, in addition to the spray-powder, at least one binder, preferably a silica binder.

The present invention is further illustrated by the following examples.

### EXAMPLES

### Example 1

### Preparation of a Titanium Containing Zeolitic Material having an MWW Framework Structure (Ti-MWW)

### Example 1.1

### Synthesis of the Boron-Containing MWW (B-MWW)

### a) Hydrothermal Synthesis

480 kg de-ionized water were provided in a vessel. Under stirring at 70 rpm (rounds per minute), 166 kg boric acid were suspended in the water. The suspension was stirred for another 3 h. Subsequently, 278 kg piperidine were added, and the mixture was stirred for another hour. To the resulting solution, 400 kg Ludox® AS-40 were added, and the resulting mixture was stirred at 70 rpm for another hour.

In this synthesis mixture, the boron source boric acid, calculated as elemental boron, relative to the silicon source Ludox® AS-40, calculated as elemental silicon, was present in a molar ratio of 1:1; the water relative to the silicon source Ludox® AS-40, calculated as elemental silicon, was present in a molar ratio of 10:1; and the template compound piperidine relative to the silicon source Ludox® AS-40, calculated as elemental silicon, was present in a molar ratio of 1.2:1.

The finally obtained mixture was transferred to a crystallization vessel and heated to 175° C. within 5 h under autogenous pressure and under stirring (50 rpm). The temperature of 175° C. was kept essentially constant for 60 h; during these 60 h, the mixture was stirred at 50 rpm. Subsequently, the mixture was cooled to a temperature of from 50-60° C. within 5 h.

The mother liquor containing the crystallized BMWW precursor had a pH of 11.3 as determined via measurement with a pH electrode.

### b) pH Adjustment

To the mother liquor obtained in a), 1400 kg of a 10 weight-% HNO₃ aqueous solution were added under stirring at 50 rpm (rounds per minute). The adding was carried out at a temperature of the suspension of 40° C. After the addition of the 10 weight-% HNO3 aqueous solution, the resulting suspension was further stirred for 5 h under stirring at 50 rpm at a temperature of the suspension of 40° C. The pH of the thus pH-adjusted mother liquor as determined via measurement with a pH electrode was 7.

### c) Spray-Drying and Calcination

From the pH-adjusted mother liquor obtained in b), the B-MWW precursor was separated by filtration using different types of filtration devices (suction filter with filter material Sefar Tetex® Mono 24-1100-SK 012, centrifugal filter, candle filter). The filter cake was then washed with deionized water until the washing water had a conductivity of less then 700 microSiemens/cm. From the washed filter cake, an aqueous suspension was prepared having a solids content of 15 weight-%. The suspension was subjected to spray-drying in a spray-tower with the following spray-drying conditions:

| | |
|---|---|
| drying gas, nozzle gas: | technical nitrogen |
| temperature drying gas: | |
| temperature spray tower (in): | 270-340° C. |
| temperature spray tower (out): | 150-167° C. |
| temperature filter (in): | 140-160° C. |
| temperature scrubber (in): | 50-60° C. |
| temperature scrubber (out): | 34-36° C. |
| pressure difference filter: | 8.3-10.3 mbar |
| nozzle: | |
| two-component nozzle | supplier Gerig; size 0 |
| nozzle gas temperature: | room temperature |
| nozzle gas pressure: | 2.5 bar |
| operation mode: | nitrogen straight |
| apparatus used: | spray tower with one nozzle |
| configuration: | spray tower - filter - scrubber |
| gas flow: | 1900 kg/h |
| filter material: | Nomex ® needle-felt 20 m² |
| dosage via flexible tube pump: | SP VF 15 (supplier: Verder) |

The spray tower was comprised of a vertically arranged cylinder having a length of 2,650 mm, a diameter of 1,200 mm, which cylinder was conically narrowed at the bottom. The length of the conus was 600 mm. At the head of the cylinder, the atomizing means (a two-component nozzle) were arranged. The spray-dried material was separated from the drying gas in a filter downstream of the spray tower, and the drying gas was then passed through a scrubber. The suspension was passed through the inner opening of the nozzle, and the nozzle gas was passed through the ring-shaped slit encircling the opening.

The spray-dried material was then subjected to calcination at 650° C. in a rotary calciner with a throughput in the range of from 0.8 to 1.0 kg/h.

The obtained BMWW had a boron content of 1.3 weight-%, a silicon content of 44 weight-%, and a total organic carbon (TOC) content of less than 0.1 weight-% and a crystallinity of 88%, determined by XRD. The BET specific surface area determined via nitrogen adsorption at 77 K according to DIN 66131 was 468 m²/g.

### Example 1.2

### Preparation of Deboronated Zeolitic Material having an MWW Framework Structure

### a) Deboronation

1590 kg water was passed into a vessel equipped with a reflux condenser. Under stirring at 40 rpm, 106 kg of the spray-dried material obtained according to section 1.1 were suspended into the water. Subsequently, the vessel was closed and the reflux condenser put into operation. The stirring rate was increased to 70 rpm under stirring at 70 rpm, the content of the vessel was heated to 100° C. within 10 h and kept at this temperature for 10 h. Then, the content of the vessel was cooled to a temperature of less than 50° C. The resulting deboronated zeolitic material of structure type MWW was separated from the suspension by filtration under a nitrogen pressure of 2.5 bar and washed four times with deionized water. After the filtration, the filter cake was dried in a nitrogen stream for 6 h. The obtained deboronated zeolitic had a residual moisture content of 80%, as determined using an IR (infrared) scale at 160° C.

### b) Spray-Drying

From the nitrogen-dried filter cake having a residual moisture content of 80% obtained according to section a) above, an aqueous suspension was prepared with deionized water, the suspension having a solid content of 15 weight-%. This suspension was subjected to spray-drying in a spray-tower with the following spray-drying conditions:

| | |
|---|---|
| drying gas, nozzle gas: | technical nitrogen |
| temperature drying gas: | |
| temperature spray tower (in): | 290-310° C. |
| temperature spray tower (out): | 140-160° C. |
| temperature filter (in): | 140-160° C. |
| temperature scrubber (in): | 40-60° C. |
| temperature scrubber (out): | 20-40° C. |
| pressure difference filter: | 6.0-10.0 mbar |
| nozzle: | |
| two-component nozzle: | supplier Niro, diameter 4 mm |
| nozzle gas pressure: | 2.5 bar |
| operation mode: | nitrogen straight |
| apparatus used: | spray tower with one nozzle |
| configuration: | spray tower - filter - scrubber |
| gas flow: | 1900 kg/h |
| filter material: | Nomex ® needle-felt 20 m² |
| dosage via flexible tube pump: | VF 15 (supplier: Verder) |

The spray tower was comprised of a vertically arranged cylinder having a length of 2,650 mm, a diameter of 1,200 mm, which cylinder was conically narrowed at the bottom. The length of the conus was 600 mm. At the head of the cylinder, the atomizing means (a two-component nozzle) were arranged. The spray-dried material was separated from the drying gas in a filter downstream of the spray tower, and the drying gas was then passed through a scrubber. The suspension was passed through the inner opening of the nozzle, and the nozzle gas was passed through the ring-shaped slit encircling the opening. The obtained spray-dried zeolitic material having an MWW framework structure had a boron content of 0.04 weight-%, a silicon content of 42 weight-%, a total organic carbon (TOC) content of less than 0.1 weight-%, and a crystallinity of 82%, determined a by XRD. The BET specific surface area determined via nitrogen adsorption at 77 K according to DIN 66131 was 462 m²/g.

### Example 1.3

### Preparation of a Titanium Containing Zeolitic Material having an MWW Framework Structure

### a) Hydrothermal Synthesis

Based on the deboronated MWW material obtained above, a zeolitic material of structure type MWW containing titanium (Ti) was prepared, referred to in the following as TiMWW.

| | | |
|---|---|---|
| Starting materials: | deionized water: | 789 g |
| | piperidine: | 291 g |
| | tetrabutylorthotitanate: | 41.4 g |
| | deboronated zeolitic material: | 192 g |

500 g of distilled water was filled in a beaker and 291g piperidine were added and the mixture was stirred for 5 min. Afterwards 41.,4 g of tetrabutylorthotitanate was added under stirring and the mixture was further stirred for 30 min before the addition of 289 g of distilled water. After stirring for another 10 min, 192 g of MWW material were added under stirring and the suspension was further stirred for another 30 min. The suspension was then transferred to an autoclave and heated in 90 min to 170° C. under stirring (100 rpm) and kept there for 48 h. The pressure increase during the synthesis is 9 bar. Subsequently, the obtained suspension containing TiMWW was cooled within 1 h below 50° C.

### b) Spray-Drying

The obtained suspension was diluted with water to have a concentration of water of 85 weight% directly subjected to spray-drying in a spray-tower with the following spray-drying conditions:

| | |
|---|---|
| drying gas, nozzle gas: | technical nitrogen |
| temperature drying gas: | |
| temperature spray tower (in): | 160-200° C. |
| temperature spray tower (out): | 150-170° C. |
| temperature filter (in): | 150-170° C. |
| temperature scrubber (in): | 30-50° C. |
| temperature scrubber (out): | 30-50° C. |
| pressure difference filter: | 6.0-10.0 mbar |
| nozzle: | |
| top-component nozzle: | supplier Niro, diameter 4 mm |
| nozzle gas pressure: | 1.5 bar |
| operation mode: | nitrogen straight |
| apparatus used: | spray tower with one nozzle |
| configuration: | spray tower - filter - scrubber |
| gas flow: | 1800 kg/h |
| filter material: | PE with PTF Membrane, Surface 1.13 m² |
| dosage via flexible tube | SP VF 15 (supplier: Verder) |
| pump: | |

The spray tower was comprised of a vertically arranged cylinder having a length of 2,650 mm, a diameter of 1,200 mm, which cylinder was conically narrowed at the bottom. The length of the conus was 600 mm. At the head of the cylinder, the atomizing means (a two-component nozzle) were arranged. The spray-dried material was separated from the drying gas in a filter downstream of the spray tower, and the drying gas was then passed through a scrubber. The suspension was passed through the inner opening of the nozzle, and the nozzle gas was passed through the ring-shaped slit encircling the opening.

### Example 1.4

### Acid Treatment of the Titanium Containing Zeolitic Material having an MWW Framework (TiMWW)

a) Acid treatment

The spray-dried TiMWW material as obtained above was subjected to acid treatment as described in the following:

| | | |
|---|---|---|
| Starting materials: | deionized water: | 1885 g |
| | nitric acid (65%) (mixed with the water | 365 g |
| | becomes 10 weight-%): | |
| | spray-dried TiMWW according to | 50 g |
| | Example 1.3: | |

1885 g deionized water were filled in a vessel. 365 g nitric acid were added, and 50 g of the spray-dried TiMWW were added under stirring. The mixture in the vessel was heated to 100° C. and kept at this temperature under autogenous pressure for 1 h under stirring (250 rpm). The thus obtained mixture was then cooled within 1 h to a temperature of less than 50° C. The cooled mixture was subjected to filtration, and the filter cake was washed with 4 L of water. After the filtration, the filter cake was dried in an oven at 120° C. for 10 h.

### b) Calcination

The dried material was then subjected to calcination at 650° C. for 5 h (heating ramp 2K/min).

The calcined material had a silicon content of 44 weight-%, a titanium content of 1.7 weight-% and a total organic carbon content of less than 0.1 weight-%. The Langmuir surface are determined via nitrogen adsorption at 77 K according to DIN 66131 was 584 m²/g, the multipoint BET specific surface area determined via nitrogen adsorption at 77 K according to DIN 66131 was 432 m²/g. The degree of crystallization determined via XRD was 84%, the average crystallite size 29.0 nm.

### Example 2

### Oxidation of 4,4′-Dichlorodiphenylsulfoxide (DCDPSO) with H₂O₂ by Use of a TiMWW Obtained According to Example 1

In a glass autoclave cooled with ice, 0.5 g of the TiMWW obtained according to Example 1 (1.4) were introduced, followed by the addition of a separately prepared solution of 60 g 1-methyl-2pyrolidone and 5.0 g DCDPSO (commercially available from Sigma-Aldrich, CAS 3085-42-5). This corresponds to about 0.8 weight-% TiMWW catalyst relative to the total amount of the obtained suspension. After the addition of the reactants the autoclave was closed and flushed with nitrogen. The suspension was stirred with a magnetic stirrer at 700 rpm and the autoclave was heated to 8° C. When the autoclave temperature reached the reaction temperature of 8° C., 10 g of an aqueous hydrogen peroxide solution (35 weight-% in water) was pumped into autoclave. After the addition of hydrogen peroxide the reaction mixture was continuously stirred for 5 hours. Subsequently, the autoclave was opened and the catalyst was removed by filtration and the reaction mixture was analyzed by GC and GC-MS.

The conversion rates of DCDPSO and the selectivity for DCDPS (4,4′-Dichlorodiphenylsulfone) in % obtained for Example 2 are summarized in Table 1 below. The conversion and selectivity were calculated according to the formulas indicated below Table 1 based on a GC analysis. A 30 m CP Sil 8 column with an internal diameter of 0.25 mm ID was used for analysis.

### Example 3

### Oxidation of 4,4′-Dichlorodiphenylsulfoxide (DCDPSO) with H₂O₂ by Use of a TiMWW Obtained According to Example 1

In a glass autoclave cooled with ice, 0.5 g of the TiMWW obtained according to Example 1 (1.4) were introduced, followed by the addition of a separately prepared solution of 60g 1-methyl-2-pyrolidone and 5.0 g DCDPSO (commercially available from Sigma-Aldrich, CAS 3085-42-5). This corresponds to about 0.8 weight-% TiMWW catalyst relative to the total amount of the obtained suspension. After the addition of the reactants the autoclave was closed and flushed with nitrogen. The suspension was stirred with a magnetic stirrer at 700 rpm and the autoclave was heated to 50° C. When the autoclave temperature reached the reaction temperature of 50° C., 10 g of an aqueous hydrogen peroxide solution (35 weight-% in water) was pumped into autoclave. After the addition of hydrogen peroxide the reaction mixture was continuously stirred for 5 hours. Subsequently, the autoclave was opened and the catalyst was removed by filtration and the reaction mixture was analyzed by GC and GC-MS.

The conversion rates of DCDPSO and the selectivity for DCDPS (4,4′-Dichlorodiphenylsulfone) in % obtained for Example 3 are summarized in Table 1 below. The conversion and selectivity were calculated according to the formulas indicated below Table 1 based on a GC analysis. A 30 m CP Sil 8 column with an internal diameter of 0.25 mm ID was used for analysis.

### Example 4

### Oxidation of 4,4′-Dichlorodiphenylsulfoxide (DCDPSO) with H₂O₂ by Use of a TiMWW Obtained According to Example 1

In a glass autoclave cooled with ice, 0.5 g of the TiMWW obtained according to Example 1 (1.4) were introduced, followed by the addition of a separately prepared solution of 60 g 1-methyl-2pyrolidone and 5.0 g DCDPSO (commercially available from Sigma-Aldrich, CAS 3085-42-5). This corresponds to about 0.8 weight-% TiMWW catalyst relative to the total amount of the obtained suspension. After the addition of the reactants the autoclave was closed and flushed with nitrogen. The suspension was stirred with a magnetic stirrer at 700 rpm and the autoclave was heated to 70° C. When the autoclave temperature reached the reaction temperature of 70° C., 10 g of an aqueous hydrogen peroxide solution (35 weight-% in water) was pumped into autoclave. After the addition of hydrogen peroxide the reaction mixture was continuously stirred for 5 hours. Subsequently, the autoclave was opened and the catalyst was removed by filtration and the reaction mixture was analyzed by GC and GC-MS.

The conversion rates of DCDPSO and the selectivity for DCDPS (4,4′-Dichlorodiphenylsulfone) in % obtained for Example 4 are summarized in Table 1 below. The conversion and selectivity were calculated according to the formulas indicated below Table 1 based on a GC analysis. A 30 m CP Sil 8 column with an internal diameter of 0.25 mm ID was used for analysis.

### Example 5

### Oxidation of 4,4′-Dichlorodiphenylsulfoxide (DCDPSO) with H₂O₂ by Use of a TiMWW Obtained According to Example 1

In a glass autoclave cooled with ice, 1.0 g of the TiMWW obtained according to Example 1 (1.4) were introduced, followed by the addition of a separately prepared solution of 60 g 1-methyl-2pyrolidone and 5.0 g DCDPSO (commercially available from Sigma-Aldrich, CAS 3085-42-5). This corresponds to about 1.5 weight-% TiMWW catalyst relative to the total amount of the obtained suspension. After the addition of the reactants the autoclave was closed and flushed with nitrogen. The suspension was stirred with a magnetic stirrer at 700 rpm and the autoclave was heated to 70° C. When the autoclave temperature reached the reaction temperature of 70° C., 10 g of an aqueous hydrogen peroxide solution (35 weight-% in water) was pumped into autoclave. After the addition of hydrogen peroxide the reaction mixture was continuously stirred for 5 hours. Subsequently, the autoclave was opened and the catalyst was removed by filtration and the reaction mixture was analyzed by GC and GC-MS.

The conversion rates of DCDPSO and the selectivity for DCDPS (4,4′-Dichlorodiphenylsulfone) in % obtained for Example 5 are summarized in Table 1 below. The conversion and selectivity were calculated according to the formulas indicated below Table 1 based on a GC analysis. A 30 m CP Sil 8 column with an internal diameter of 0.25 mm ID was used for analysis.

### Example 6

### Oxidation of 4,4′-Dichlorodiphenylsulfoxide (DCDPSO) with H₂O₂ by Use of a TiMWW Obtained According to Example 1

In a glass autoclave cooled with ice, 1.0 g of the TiMWW obtained according to Example 1 (1.4) were introduced, followed by the addition of a separately prepared solution of 60 g 1-methyl-2pyrolidone and 5.0 g DCDPSO (commercially available from Sigma-Aldrich, CAS 3085-42-5). This corresponds to about 0.8 weight-% TiMWW catalyst relative to the total amount of the obtained suspension. After the addition of the reactants the autoclave was closed and flushed with nitrogen. The suspension was stirred with a magnetic stirrer at 700 rpm and the autoclave was heated to 70° C. When the autoclave temperature reached the reaction temperature of 70° C., 7 g of an aqueous hydrogen peroxide solution (35 weight-% in water) was pumped into autoclave. After the addition of hydrogen peroxide the reaction mixture was continuously stirred for 5 hours. Subsequently, the autoclave was opened and the catalyst was removed by filtration and the reaction mixture was analyzed by GC and GC-MS.

The conversion rates of DCDPSO and the selectivity for DCDPS (4,4′-Dichlorodiphenylsulfone) in % obtained for Example 6 are summarized in Table 1 below. The conversion and selectivity were calculated according to the formulas indicated below Table 1 based on a GC analysis. A 30 m CP Sil 8 column with an internal diameter of 0.25 mm ID was used for analysis.

### Example 7

### Oxidation of 4,4′-Dichlorodiphenylsulfoxide (DCDPSO) with H₂O₂ by Use of a TiMWW Obtained According to Example 1

In a glass autoclave cooled with ice, 0.5 g of the TiMWW obtained according to Example 1 (1.4) were introduced, followed by the addition of a separately prepared solution of 60 g tetrahydrofuran and 5.0 g DCDPSO (commercially available from Sigma-Aldrich, CAS 3085-42-5). This corresponds to about 0.8 weight-% TiMWW catalyst relative to the total amount of the obtained suspension. After the addition of the reactants the autoclave was closed and flushed with nitrogen. The suspension was stirred with a magnetic stirrer at 700 rpm and the autoclave was heated to 50° C. When the autoclave temperature reached the reaction temperature of 50° C., 10 g of an aqueous hydrogen peroxide solution (35 weight-% in water) was pumped into autoclave. After the addition of hydrogen peroxide the reaction mixture was continuously stirred for 5 hours. Subsequently, the autoclave was opened and the catalyst was removed by filtration and the reaction mixture was analyzed by GC and GC-MS.

The conversion rates of DCDPSO and the selectivity for DCDPS (4,4′-Dichlorodiphenylsulfone) in % obtained for Example 7 are summarized in Table 1 below. The conversion and selectivity were calculated according to the formulas indicated below Table 1 based on a GC analysis. A 30 m CP Sil 8 column with an internal diameter of 0.25 mm ID was used for analysis.

TABLE 1

| | | Amount of | | | | |
|---|---|---|---|---|---|---|
| | | catalyst in | | | Conversion | |
| | Temp./ | reaction mixture/ | Ratio | | rate | Selectivity |
| | ° C. | weight-% | DCDPSO:H₂O | Time/h | DCDPSO/% | DCDPS/% |
| Example 2 | 8 | 0.8 | 0.2 | 5 | 64.4 | 82.5 |
| Example 3 | 50 | 0.8 | 0.2 | 5 | 47.5 | 98.6 |
| Example 4 | 70 | 0.8 | 0.2 | 5 | 42.6 | 87.2 |
| Example 5 | 70 | 1.5 | 0.2 | 5 | 77.8 | 92.1 |
| Example 6 | 70 | 0.8 | 0.2 | 5 | 48.0 | 85.3 |
| Example 7 | 50 | 0.8 | 0.2 | 5 | 16.2 | 71.6 |

The conversion rate of DCDPSO was calculated based on the following equation:

Conversion (%)=100−(mol(DCDPSO) after reaction/mol(DCDPSO) introduced)*100

The selectivity of DCDPS was calculated based on the following equation:

Selectivity (%)=(mol(DCDPS) after the reaction/mol(DCDPSO) consumed)*100

### Cited Prior Art

RU-C-2158257
CN-A-102351757
CN-A-102351756
WO-A-2012/143281
CN-A-102838516

1. A process for oxidizing a sulfoxide to a sulfone, said process comprising
(i) reacting the sulfoxide with hydrogen peroxide in the presence of a catalyst, obtaining a mixture (M) comprising the sulfone and the catalyst,
wherein the catalyst comprises a porous titanium-containing silicate as a catalytically active material.

2. The process of claim 1, wherein the sulfoxide has a structure according to formula (I) 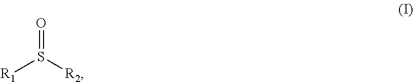
and the sulfone has a structure according to formula (II) 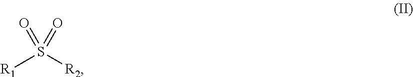
wherein R₁ and R₂ are independently from one another a linear or branched, substituted or unsubstituted alkyl residue, or a substituted or unsubstituted aryl or heteroaryl residue.

3. The process of claim 1, wherein the sulfoxide is 4,4′-dichlorodiphenylsulfoxide or 4,4′-dihydroxydiphenylsulfoxide.

4. The process of claim 1, wherein the porous titanium-containing silicate comprised in the catalyst is a titanium-containing zeolitic material having a zeolitic framework structure comprising titanium and silicon.

5. The process of claim 4, wherein the framework structure of the titanium-containing zeolitic material comprised in the catalyst is an MWW-type framework structure.

6. The process of claim 5, wherein the framework structure of the titanium-containing zeolitic material comprised in the catalyst has a titanium content in a range of from 0.5 to 3.0 weight-%, calculated as element and based on a total weight of the titanium-containing zeolitic material, and a silicon content in a range of from 30 to 50 weight-%, calculated as element and based on the total weight of the titanium-containing zeolitic material.

7. The process of claim 1, wherein the hydrogen peroxide used in (i) is employed as an aqueous hydrogen peroxide solution, having a hydrogen peroxide content in a range of from 10 to 70 weight-%, based on a total weight of the aqueous solution.

8. The process of claim 1, wherein at the beginning of said reacting (i), a molar ratio of hydrogen peroxide to the sulfoxide is in a range of from 1:1 to 50:1.

9. The process of claim 1, wherein at the beginning of said reacting (i), a molar ratio of the sulfoxide to titanium contained in the titanium-containing silicate is in a range of from 10:1 to 500:1.

10. The process of claim 1, wherein said reacting (i) is carried out in the presence of a solvent and the mixture (M) additionally comprises the solvent.

11. The process of claim 10, wherein the solvent is selected from the group consisting of 1-methyl-2-pyrrolidone, tetrahydrofuran, dioxane, a chlorinated hydrocarbon, and a combination of two or more thereof.

12. The process of claim 11, wherein at the beginning of said reacting (i), a molar ratio of the sulfoxide to the solvent is in a range of from 0.01:1 to 10:1.

13. The process of claim 1, wherein said reacting (i) is carried out in the presence of at least one inert gas.

14. The process of claim 1, wherein said reacting (i) is carried out at a temperature in a range of from 0 to 90° C.

15. The process of claim 1, wherein said reacting (i) is carried out under a pressure of at most 15 bar.

16. The process of claim 1, wherein said reacting (i) is carried out in a batch mode.

17. The process of claim 16, wherein to said reacting (i) is carried out for a period of time in a range of from 1 to 15 h.

18. The process of claim 1, further comprising
(ii) separating the catalyst from the mixture (M).

19. The process of claim 2, further comprising
(iii) separating the sulfone according to formula (II) from the mixture (M) preferably by precipitation.

20. The process of claim 1, wherein the catalyst is a spray-powder.
